(19) United States
(12) Patent Application Publication
(10) Pub. No.: US 2009/0101514 A1
(43) Pub. Date: Apr. 23, 2009

(54) Electrodeposition Method for Metals

(75) Inventors: Toshiyuki Nohira, Kyoto-shi (JP); Rika Hagiwara, Kyoto-shi (JP); Jun Shimano, Kyoto-shi (JP)

Correspondence Address: KRATZ, QUINTOS & HANSON, LLP, 1420 K Street, N.W., Suite 400, WASHINGTON, DC, 20005, US

(73) Assignee: KYOTO UNIVERSITY, Kyoto -shi (JP)

(21) Appl. No.: 11/991,501

(22) PCT Filed: Sep. 5, 2006
(86) PCT No.: PCT/JP2006/317488

§ 371 Date: Mar. 5, 2008

(30) Foreign Application Priority Data
Sep. 7, 2005 (JP) 2005-259827

(51) Int. Cl. C25D 3/66; C07D 207/06

(52) U.S. Cl. 205/232; 205/230; 548/579

(57) ABSTRACT

An objective of the present invention is to provide an electrodeposition method for metals using a molten salt, which easily enables the electrodeposition of various types of metals such as refractory metals and rare earth metals. In order to solve this problem, the invention is characterized in that it is effected at the electrodeposition temperature in a range of from 100° C. to 200° C. using a molten salt of quaternary ammonium halide represented by the general formula (I) below (wherein, in the formula, R¹, R², R³, and R⁴, which may be the same or different from each other and may have a substituent, each represents an alkyl group having 1 to 12 carbon atoms or a cycloalkyl group having 5 to 7 carbon atoms; and X⁻ represents a halide anion which is a counter-ion of quaternary ammonium cation) and/or a molten salt of pyrrolidinium halide represented by the general formula (II) below (wherein, in the formula, R⁵ and R⁶, which may be the same or different from each other and may have a substituent, each represents an alkyl group having 1 to 12 carbon atoms or a cycloalkyl group having 5 to 7 carbon atoms; and X⁻ represents a halide anion which is a counter-ion of pyrrolidinium cation).

[Chemical 1]

R¹R²R³R⁴N⁺X⁻ (I)

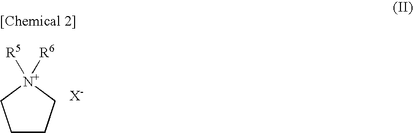

### TECHNICAL FIELD

The present invention relates to an electrodeposition method for metals using a molten salt.

### BACKGROUND ART

It is well known that various methods using a molten salt (a liquid produced by melting a salt) are proposed as the methods for the electrodeposition of a metal; there is proposed, for instance, a method described in patent document 1, which comprises using an ambient temperature molten salt made from an organic quaternary ammonium cation such as tetraalkylammonium cation and a fluorine-based anion such as [CF₃(CH₂)ₙSO₂]₂N⁻ (wherein n represents an integer greater than or equal to 0), and after dissolving a metallic salt therein, effecting an electrodeposition process under a temperature condition of from 0° C. to 100° C. However, refractory metals having a melting point of 1500° C. or higher, such as Ti, V, Cr, Zr, Nb, Mo, Hf, Ta, and W, or rare earth metals such as Nd and Sm, which abound in industrial applications, yield ions that are electrochemically stable in the molten salt at a room temperature. Accordingly, if the electrodeposition of such metals is attempted at 100° C. or lower using the ambient temperature molten salt in accordance with the method described in patent document 1, it is often the case that the decomposition of the molten salt occurs in preference to the deposition of the object metal intended for the electrodeposition on the cathode. Furthermore, it is more likely that the metal used for the anode, which is intended for the electrodeposition, does not dissolve efficiently as an ion at the anode. As a result, if continuous electrodeposition is attempted, there occurs a problem that the molten salt undergoes deterioration. In the case of the method described in patent document 1, in particular, the aforementioned refractory metals and rare earth metals cannot be easily dissolved as ions at the anode, but electrolytic oxidation of the organic cation constituting the molten salt occurs as an anodic reaction. The electrolytic oxidation of the organic cation is attributed to the fact that the fluorine-based anion is less apt to be electrolytically oxidized as compared with the organic cation. Once the organic cation is decomposed by electrolytic oxidation, the decomposition products derived from the organic cations accumulate in the system to make the molten salt no longer feasible for the electrodeposition.

Because the electrochemical stability of the ions of refractory metals and rare earth metals decreases with elevating temperature, these metals tend to be easily deposited by elevating the electrodeposition temperature. Accordingly there are known methods, for example, performing the electrodeposition of such metals at a temperature as high as 350° C. or higher using inorganic molten salts such as a ZnBr₂—NaBr based molten salt and a ZnCl₂—NaCl based molten salt (see non-patent document 1 and non-patent document 2), however, the use of these methods are limited because the materials for constructing the apparatus and the electrode materials are restricted to materials resistant to high temperatures, such as metals and ceramics. Thus, the present inventors proposed a method for electrodepositing tungsten using a ZnCl₂—NaCl—KCl based molten salt in non-patent document 3. However, because the melting point of this inorganic molten salt is 203° C., the electrodeposition temperature must be set at a temperature higher than or equal to the melting point. Accordingly, although this method is superior to the above methods using inorganic molten salts in the point that it can be effected at a lower temperature, it is still necessary to develop a method feasible at a more lower temperature, which enables long-term use of a wide variety of materials to be used as the materials for constructing the apparatus and the electrode materials.

Patent document 1: JP-A-2002-371397.
Non-patent document 1: Denki Kagaku oyobi Kogyo Butsuri Kagaku, 56, 40 (1988).
Non-patent document 2: J. Electrochem. Soc., 138, 767 (1991).
Non-patent document 3: Electrochemical and Solid-State Letters, 8(7) C91 (2005).

### DISCLOSURE OF THE INVENTION

### Problems that the Invention is to Solve

Accordingly, an objective of the present invention is to provide an electrodeposition method for metals using a molten salt, which easily enables the electrodeposition of various types of metals such as refractory metals and rare earth metals.

### Means for Solving the Problems

In the light of the aforementioned circumstances, the present inventors have intensively studied, and as a result, they have found that the electrodeposition of various types of metals such as refractory metals and rare earth metals can be easily conducted using some kind of a molten salt of quaternary ammonium halide and a molten salt of pyrrolidinium halide at an electrodeposition temperature in a range of from 100° C. to 200° C.

An electrodeposition method for metals using a molten salt of the present invention, which has been accomplished on the basis of the findings above, as claimed in claim 1, is characterized in that it is effected at the electrodeposition temperature in a range of from 100° C. to 200° C. using a molten salt of quaternary ammonium halide represented by the general formula (I) below (wherein, in the formula, R¹, R², R³, and R⁴, which may be the same or different from each other and may have a substituent, each represents an alkyl group having 1 to 12 carbon atoms or a cycloalkyl group having 5 to 7 carbon atoms; and X⁻ represents a halide anion which is a counter-ion of quaternary ammonium cation) and/or a molten salt of pyrrolidinium halide represented by the general formula (II) below (wherein, in the formula, R⁵ and R⁶, which may be the same or different from each other and may have a substituent, each represents an alkyl group having 1 to 12 carbon atoms or a cycloalkyl group having 5 to 7 carbon atoms; and X⁻ represents a halide anion which is a counter-ion of pyrrolidinium cation).

[Chemical 4]

R¹R²R³R⁴N⁺X⁻ (I)

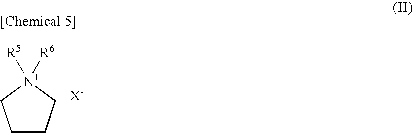

Furthermore, the method as described in claim 2 is the method as claimed in claim 1, which is characterized in that the halide anion is a chloride ion.

Further, the method as described in claim 3 is the method as claimed in claim 1, which is characterized in that a metal halide compound is dissolved in the molten salt.

In addition, the method as described in claim 4 is the method as claimed in claim 3, which is characterized in that the metal halide compound is at least one type selected from the group consisting of zinc chloride, tin chloride, and iron chloride.

Additionally, the method as described in claim 5 is the method as claimed in claim 3, which is characterized in that 0.5 mol to 2 mol of the metal halide compound is dissolved in 1 mol of the molten salt.

Furthermore, the method as described in claim 6 is the method as claimed in claim 1, which is characterized in that an alkali metal chloride and/or an alkali metal fluoride is added in the molten salt.

Further, the method as described in claim 7 is the method as claimed in claim 1, which is characterized in that the electrodeposition temperature is in a range of from 130° C. to 180° C.

In addition, the method as described in claim 8 is the method as claimed in claim 1, which is characterized in that the object metal intended for the electrodeposition is at least one type selected from a group consisting of refractory metals having a melting point of 1500° C. or higher, rare earth metals, and alloys containing at least one metal thereof.

Additionally, a molten salt of pyrrolidinium halide of the present invention is, as claimed in claim 9, characterized in that it is represented by the general formula (II) below (wherein, in the formula, R⁵ and R⁶, which may be the same or different from each other and may have a substituent, each represents an alkyl group having 1 to 12 carbon atoms or a cycloalkyl group having 5 to 7 carbon atoms; and X⁻ represents a halide anion which is a counter-ion of pyrrolidinium cation).

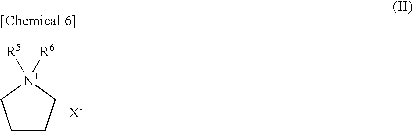

### EFFECT OF THE INVENTION

In accordance with the present invention, it is possible to provide an electrodeposition method for metals using a molten salt, which easily enables the electrodeposition of various types of metals including refractory metals having a melting point of 1500° C. or higher, such as Ti, V, Cr, Zr, Nb, Mo, Hf, Ta, and W, or rare earth metals such as Nd and Sm. By thus enabling the electrodeposition of such refractory metals at a temperature of 200° C. or lower by the invention, the method can be utilized as a part of the next generation microfabrication technology by applying to Galvanoformung (electroforming) in LIGA (Lithographie, Galvanoformung, Abformung) process. Furthermore, by enabling the electrodeposition of rare earth metals, novel production method can be provided for functional materials such as magnetic materials, semiconductor materials, and hydrogen absorbing materials.

### BRIEF DESCRIPTION OF THE DRAWINGS

FIG. 1 shows a DSC curve of TriMePeAmCl—ZnCl₂ (molar ratio of 50:50) according to an example, obtained at a heating rate of 10° C. min⁻¹.

FIG. 2 shows a DSC curve of EtMePyrCl—ZnCl₂ (molar ratio of 50:50) according to an example, obtained at a heating rate of 10° C. min⁻¹.

FIG. 3 shows a cyclic voltammogram of TriMePeAmCl—ZnCl₂ (molar ratio of 50:50) according to an example, obtained on the cathode side at a potential scan rate of 10 mVs⁻¹.

FIG. 4 shows a cyclic voltammogram of TriMePeAmCl—ZnCl₂ (molar ratio of 50:50) according to an example, obtained on the anode side at a potential scan rate of 10 mVs⁻¹.

### BEST MODE FOR CARRYING OUT THE INVENTION

The electrodeposition method for metals using a molten salt of the present invention is characterized in that it is effected at the electrodeposition temperature in a range of from 100° C. to 200° C. using a molten salt of quaternary ammonium halide represented by the general formula (I) below (wherein, in the formula, R¹, R², R³, and R⁴, which may be the same or different from each other and may have a substituent, each represents an alkyl group having 1 to 12 carbon atoms or a cycloalkyl group having 5 to 7 carbon atoms; and X⁻ represents a halide anion which is a counter-ion of quaternary ammonium cation) and/or a molten salt of pyrrolidinium halide represented by the general formula (II) below (wherein, in the formula, R⁵ and R⁶, which may be the same or different from each other and may have a substituent, each represents an alkyl group having 1 to 12 carbon atoms or a cycloalkyl group having 5 to 7 carbon atoms; and X⁻ represents a halide anion which is a counter-ion of pyrrolidinium cation).

[Chemical 7]

R¹R²R³R⁴N⁺X⁻ (I)

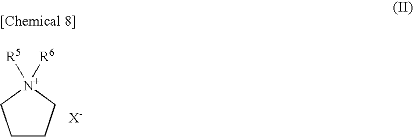

In the molten salt of quaternary ammonium halide represented by the general formula (I) and the molten salt of pyrrolidinium halide represented by the general formula (II), the alkyl group stated as an alkyl group having 1 to 12 carbon atoms, which may have a substituent, may be in the form of a straight chain or a branched chain; more specifically, there can be mentioned methyl group, ethyl group, n-propyl group, isopropyl group, n-butyl group, isobutyl group, sec-butyl group, tert-butyl group, n-pentyl group, isopentyl group, n-hexyl group, n-octyl group, n-decyl group, and n-dodecyl group. These alkyl groups may have a substituent such as hydroxyl group, amino group, cyano group, nitro group, and a halogen. Examples for the cycloalkyl group stated as a cycloalkyl group having 5 to 7 carbon atoms, which may have a substituent, include cyclopentyl group, cyclohexyl group, and cycloheptyl group. These cycloalkyl groups may have a substituent which include the same substituents enumerated for the alkyl groups and alkyl groups having 1 to 6 carbon atoms.

As the halide anion stated as the counter-ion of the organic cation (quaternary ammonium cation and pyrrolidinium cation), there may be mentioned chloride ion, bromide ion, and iodide ion. By employing these anions as the halide anion, even if the molten salt undergoes decomposition when electrolysis is carried out on the cathode, the electrolytic oxidation of these anions can be effected in preference to the electrolytic oxidation of the organic cations on the anode. Hence, the decomposition products can be discharged out of the system in the form of gaseous halogen. Accordingly, by additionally feeding into the system a metal halide compound corresponding to the halide anion, the electrodeposition can be effected for a long time. In addition, preferred as the halide anion is the chloride ion which is characterized in that it has high ion conductivity as a molten salt and that it is easily released out of the system in the form of gaseous halogen.

Furthermore, the quaternary ammonium halide represented by the general formula (I) and the pyrrolidinium halide represented by the general formula (II) can be synthesized by a method known in the art.

A metal halide compound may be dissolved in the molten salt. In a case where in the metal halide compound, a halide anion is a chloride ion, there can be mentioned zinc chloride, tin chloride, and iron chloride, however, preferred among them is zinc chloride which is characterized in that it has a wide reduction potential window and that it is capable of favorably depositing refractory metals. By dissolving in molten salt, the metal halide compounds constitute halide metal complex anions (such as ZnCl₃⁻, SnCl₃⁻, and FeCl₄⁻) as counter-ions of the organic cations, which, in general, provide effects of lowering the melting point of the molten salt while increasing the decomposition temperature. Preferably, the metal halide compound is dissolved in an amount of from 0.5 mol to 2 mol to 1 mol of the molten salt. If the dissolved amount is too small, the desired effect may not be obtained. If the dissolved amount is too large, on the other hand, the characteristics of the metal halide compound itself may appear strongly to lead an increase in the melting point or suppress the deposition of the object metal intended for the deposition. The metal halide compound is preferably used in the form of an anhydride. In the case it is used in the form of a hydrate, the electrolysis of water attributed to the hydrate occurs, and there are fears of lowering current efficiency, hindering the deposition of the object metal intended for the electrodeposition, or lowering the quality of the deposit due to the inclusion of hydrogen.

The electrodeposition method for metals of the invention can be carried out, for instance, using an apparatus which employs a three electrode method well known in the art (if necessary, reference can be made to the patent document 1 and non-patent document 3). More specifically, the raw material of the object metal intended for the electrodeposition (examples include a metal halide compound, a metal oxide compound, a metal oxyhalide compound, and a complex salt obtained by reacting one of them with an alkali metal halide compound or an alkali metal oxide compound) is dissolved in a molten salt, and the electric current is applied at a temperature in a range of from 100° C. to 200° C. In this manner, the ion solubility and the ion conductivity of the object metal intended for the electrodeposition in the molten salt are increased and the viscosity of the molten salt is reduced, as compared with the case of effecting the electrodeposition at 100° C. or lower using an ambient temperature molten salt. As a result, a higher current density can be obtained to improve the efficiency of the electrodeposition. Furthermore, the smaller the crystallite size of the electrodeposited metal, the better properties, such as strength, can be obtained. Since the rate of crystal growth in the electrodeposition increases with elevating temperature, it is difficult to deposit metals having smaller crystallite size at such a high electrodeposition temperature as those described in the methods disclosed in the non-patent documents 1 to 3. However, by setting the electrodeposition temperature in a range of from 100° C. to 200° C., it becomes easier to deposit metals having smaller crystallite size which possess superior properties. Needless to say, by taking the melting point of the thus employed molten salt into consideration, the electrodeposition temperature is set at a temperature not lower than the melting point. Considering ease in handling, in general, the temperature is preferably set in a range of from 130° C. to 180° C. Preferably, in 1 mol of the molten salt, the raw material of the object metal intended for the electrodeposition is dissolved in an amount of from 0.005 mol to 2 mol. The electrodeposition may be effected by potentiostatic electrolysis, or by galvanostatic electrolysis. In the case of carrying out potentiostatic electrolysis, the potential is preferably set in a range of from 0 to +1.0V vs. Mⁿ⁺/M (where Mⁿ⁺/M represents the redox pair of the metal deposited at the cathode limit in the molten salt and the metallic ion). In the case of carrying out galvanostatic electrolysis, the current density is preferably set in a range of from 0.1 mA/cm² to 100 mA/cm². In the case of performing the electrodeposition in an industrial scale, it is preferred to employ the galvanostatic electrolysis which can be realized with a simpler construction of the apparatuses.

In the molten salt, there may be added an alkali metal chloride such as LiCl, NaCl, and KCl, or an alkali metal fluoride such as LiF, NaF, and KF. The addition of these compounds into the molten salt enables increasing the ion solubility of the object metal intended for the electrodeposition, ameliorating the quality of the electrodeposition products, increasing the electrical conductivity of the molten salt, and the like. Thus, it facilitates the electrodeposition of, for example, refractory metals, rare-earth metals, and alloys containing at least one metal thereof; at the same time, it enables the deposition of the targeted metals as a film-like product which contains little impurities and is suitable for, for instance, electroforming in LIGA process and a coating of an object. The amount of adding the alkali metal chloride or the alkali metal fluoride into the molten salt is preferably set in a range of from ½ times to 2 times the saturation amount of the dissolution of the compound in the molten salt.

### EXAMPLES

The present invention is explained in detail by way of examples below, but it should be understood that the invention is not only limited thereto.

### Example 1

### (1) Synthesis of a Molten Salt of Quaternary Ammonium Halide Represented by the General Formula (I)

As a representative example, a method for synthesizing trimethylpentylammonium chloride (TriMePeAmCl) is described below. First, trimethylamine (Tokyo Chemical Industry Co., Ltd.; 28% in water) was mixed with 1-chloropentane (Tokyo Chemical Industry Co., Ltd.; 99%) in acetonitrile, and the mixture was stirred at 80° C. for 24 hours or longer. Then, the product was distilled and dried in vacuum at 80° C. for 24 hours or longer to obtain the desired product as a white powder. Various types of trimethylalkylammonium chloride (TriMeAlkAmCl) and tetraalkylammonium chloride (TetAlkAmCl) were synthesized similarly (except for TetBuAmCl, which was dried in vacuum at 60° C.). Thus synthesized molten salts are shown in Table 1 together with their melting points and decomposition temperatures. The melting point was determined by the result of studying the thermal behavior with elevating temperature using a differential scanning calorimetry (DSC), and the measured results obtained by a melting point apparatus. The decomposition temperature was determined by the result of studying the thermal behavior with elevating temperature using a simultaneous differential thermal analysis and thermogravimetry (DTA-TG) (the same applies for all the products hereinafter). As is clearly from Table 1, TriMeHepAmCl, TetEtAmCl, and TetPrAmCl can be stably used, for instance, at the temperature in a range of from 150° C. to 200° C., and TetBuAmCl can be stably used, for instance, at the temperature in a range of from 100° C. to 150° C.

### (2) Synthesis of a Molten Salt of Pyrrolidinium Halide Represented by the General Formula (II)

A method for synthesizing N-ethyl-N-methylpyrrolidinium chloride (EtMePyrCl) is described below. First, N-methylpyrrolidine (Sigma-Aldrich Corp.) was placed inside a pressure resistant bottle together with acetonitrile, and was cooled with liquid nitrogen. Chloroethane (Wako Pure Chemical Industries, Ltd.) was blown thereto to mix while gradually elevating temperature, and the mixture was stirred at 80° C. for 24 hours or longer. Then, the product was distilled and dried in vacuum at 80° C. for 24 hours or longer to obtain the desired product as a white powder. The melting point and the decomposition temperature of EtMePyrCl are given in Table 1. As is clearly from Table 1, EtMePyrCl can be stably used, for instance, at the temperature in a range of from 150° C. to 200° C.

TABLE 1

| | | | Melting | Decomposition |
|---|---|---|---|---|
| No. | Molten salt | Abbreviation | Point (° C.) | Temp. (° C.) |
| 1 | Trimethylpropylammonium chloride | TriMePrAmCl | 206 | 248 |
| 2 | Trimethylbutylammonium chloride | TriMeBuAmCl | 223 | 233 |
| 3 | Trimethylpentylammonium chloride | TriMePeAmCl | 198 | 224 |
| 4 | Trimethylhexylammonium chloride | TriMeHexAmCl | 193 | 227 |
| 5 | Trimethylheptylammonium chloride | TriMeHepAmCl | 137 | 223 |
| 6 | Tetramethylammonium chloride | TetMeAmCl | 266 | 344 |
| 7 | Tetraethylammonium chloride | TetEtAmCl | 123 | 262 |
| 8 | Tetrapropylammonium chloride | TetPrAmCl | 129 | 222 |
| 9 | Tetrabutylammonium chloride | TetBuAmCl | 73 | 185 |
| 10 | N-ethyl-N-methylpyrrolidinium chloride | EtMePyrCl | 145 | 265 |

### (3) Synthesis of a Mixed Molten Salt with Zinc Chloride

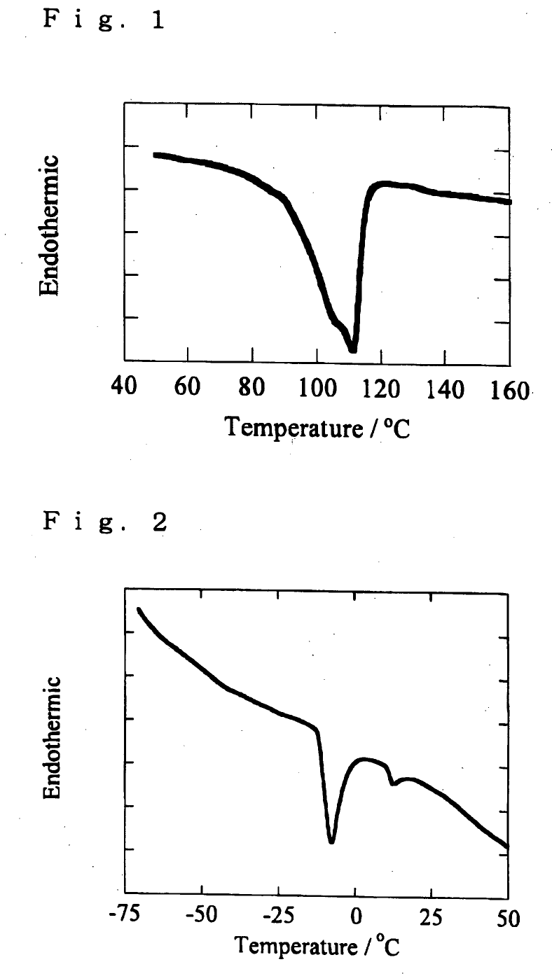

In each of the molten salts of quaternary ammonium halide represented by the general formula (I) and the molten salts of pyrrolidinium halide represented by the general formula (II), anhydrous zinc chloride (ZnCl₂) (Wako Pure Chemical Industries, Ltd.; 99.9%) was dissolved to yield a mixture at a predetermined molar ratio (50:50 or 40:60) and to synthesize the desired product. The melting point and the decomposition temperature of the thus obtained mixed molten salts are given in Table 2. The DSC curves of TriMePeAmCl—ZnCl₂ (molar ratio of 50:50) and EtMePyrCl—ZnCl₂ (molar ratio of 50:50) are each given in FIGS. 1 and 2, respectively, as representative examples. As is clearly from Table 2, except for TetEtAmCl, TetPrAmCl, TetBuAmCl, the melting point greatly decreases while the decomposition temperature increases by dissolving zinc chloride in the molten salt. Furthermore, it has been found that thus obtained mixed molten salts can be used stably at the temperature in a range of from 130° C. to 200° C., except for TetMeAmCl—ZnCl₂.

TABLE 2

| | | Molar ratio | | |
|---|---|---|---|---|
| | | of molten | Melting | Decomposition |
| No. | Molten salt | salt:ZnCl₂ | Point (° C.) | Temp. (° C.) |
| 1 | TriMePrAmCl | 50:50 | 107 | 337 |
| | | 40:60 | 87 | 340 |
| 2 | TriMeBuAmCl | 50:50 | 117 | 341 |
| | | 40:60 | 71 | 344 |
| 3 | TriMePeAmCl | 50:50 | 91 | 342 |
| | | 40:60 | 68 | 344 |
| 4 | TriMeHexAmCl | 50:50 | 102 | 343 |
| | | 40:60 | 66 | 346 |
| 5 | TriMeHepAmCl | 50:50 | 96 | 344 |
| | | 40:60 | 65 | 347 |
| 6 | TetMeAmCl | 50:50 | 170 | 400 |
| | | 40:60 | 151 | 391 |
| 7 | TetEtAmCl | 50:50 | 123 | 338 |
| | | 40:60 | 110 | 330 |
| 8 | TetPrAmCl | 50:50 | 129 | 302 |
| | | 40:60 | 115 | 305 |
| 9 | TetBuAmCl | 50:50 | 73 | 287 |
| | | 40:60 | 66 | 282 |
| 10 | EtMePyrCl | 50:50 | 38 | 280 |
| | | 40:60 | 5 | 312 |

### (4) Electrochemical Measurement of a Mixed Molten Salt Obtained from a Molten Salt of Quaternary Ammonium Halide Represented by the General Formula (I) and Zinc Chloride

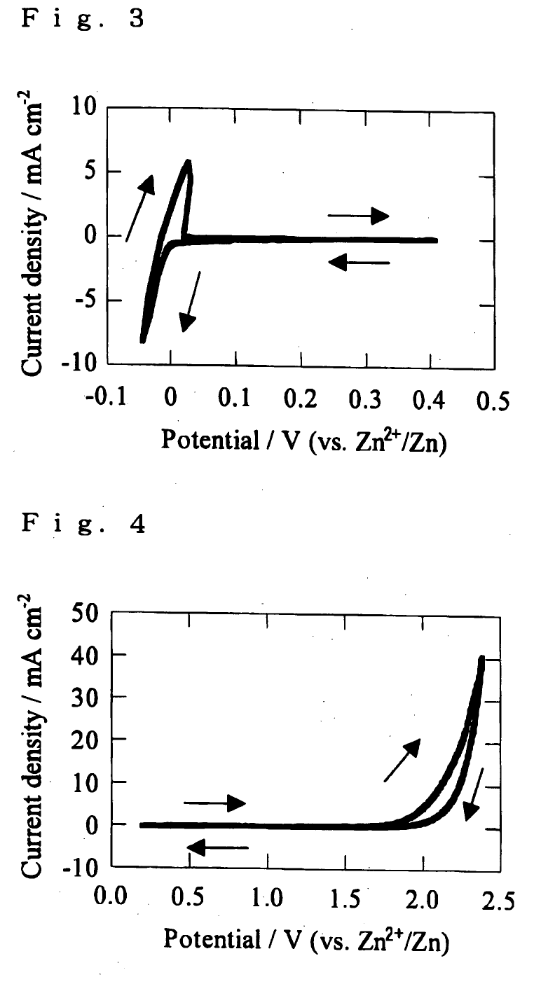

TriMePeAmCl—ZnCl₂ (molar ratio of 50:50) was selected as a representative example. About 30 ml of the molten salt was placed inside a Pyrex (Registered Trademark) beaker, and was heated by a hot stirrer at a bath temperature of 150° C. Measurement was conducted by three electrode method. A molybdenum wire (The Nilaco Corporation; 99.95%, 1 mm in diameter×5 mm in length) or a glassy carbon (Tokai Carbon Co., Ltd.; 5 mm in diameter×10 mm in length) was used as the working electrode (cathode). A nickel plate (The Nilaco Corporation; 99.7%, 10 mm in length×5 mm in width×0.2 mm in thickness) or a glassy carbon was used as the counter electrode (anode). A zinc wire (The Nilaco Corporation; 99.99%, 1 mm in diameter×5 mm in length) was used as the reference electrode. The redox potential of zinc (Zn²⁺/Zn) was taken as the standard. The handling of the molten salt and the electrochemical measurement were all conducted in a glove box under argon atmosphere. FIG. 3 shows a cyclic voltammogram obtained by potential scanning to the cathode side in the case of using a molybdenum wire as the working electrode and a glassy carbon as the counter electrode. A cathodic current was observed from around 0 V (vs. Zn²⁺/Zn), and a corresponding anodic current was also observed. Because the cathodic current observed herein was speculated to correspond to the electrodeposition of zinc, a sample of the massive electrodeposition product on the working electrode was obtained by conducting galvanostatic electrolysis (at a current density of 10 mA/cm²) for 3 hours. The sample was analyzed by X-ray photoelectron spectroscopy (XPS), and as a result, it has been confirmed to be metallic zinc (atomic composition: 99.3 atomic % of zinc, 0.3 atomic % of oxygen, and 0.4 atomic % of others). FIG. 4 shows a cyclic voltammogram obtained by potential scanning to the anode side in the case of using a glassy carbon as the working electrode and a nickel plate as the counter electrode. A rise in an anodic current was observed from around 2.0 V (vs. Zn²⁺/Zn), but no current corresponding to the reverse reaction was observed. The anodic current was speculated to correspond to the generation of gaseous chlorine. From the results above, it has been found that the potential window of TriMePeAmCl—ZnCl₂ (molar ratio of 50:50) is about 2.0V at 150° C., and that the electrodeposition of metallic zinc can be conducted according to this method.

### (5) Electrodeposition of Metallic Zinc by Galvanostatic Electrolysis Using a Mixed Molten Salt Obtained from a Molten Salt of Pyrrolidinium Halide Represented by the General Formula (II) and Zinc Chloride

Massive deposition product of metallic zinc (atomic composition: 99.2 atomic % of zinc, 0.4 atomic % of oxygen, and 0.4 atomic % of others) was obtained in the same manner as above by galvanostatic electrolysis using EtMePyrCl—ZnCl₂ (molar ratio of 50:50).

### Example 2

In TriMePeAmCl—ZnCl₂ (molar ratio of 50:50) was dissolved 0.1 mol of tungsten tetrachloride (WCl₄) with respect to 1 mol of TriMePeAmCl, and massive deposition product of metallic tungsten (atomic composition: 97.2 atomic % of tungsten, 1.5 atomic % of oxygen, and 1.3 atomic % of others) was obtained in the same manner as in Example 1 by galvanostatic electrolysis, except that the current was applied at a current density of 0.5 mA/cm².

### Example 3

In EtMePyrCl—ZnCl₂ (molar ratio of 50:50) was dissolved 0.1 mol of WCl₄ with respect to 1 mol of EtMePyrCl, and massive deposition product of metallic tungsten (atomic composition: 97.0 atomic % of tungsten, 1.6 atomic % of oxygen, and 1.4 atomic % of others) was obtained in the same manner as in Example 1 by galvanostatic electrolysis, except that the current was applied at a current density of 0.5 mA/cm².

### Example 4

### Electrodeposition by Potentiostatic Electrolysis

### Experiment A:

TriMePeAmCl was dried in vacuum at 120° C. for 24 hours. Further, ZnCl₂ and KF were dried in vacuum at 200° C. for 24 hours. TriMePeAmCl and ZnCl₂ were weighed at a molar ratio of 50:50 in a glove box under argon atmosphere, and were placed inside an alumina crucible. Then, 2 mol of KF (which approximately corresponds to the saturation amount of the dissolution of the compound in the molten salt) and 0.5 mol of WCl₄ were weighed with respect to 100 mol of a mixture of TriMePeAmCl and ZnCl₂, and were placed inside the alumina crucible into which TriMePeAmCl and ZnCl₂ were placed as above. Subsequently, the above alumina crucible into which the raw material powder were placed was heated to 150° C. inside the same glove box as above to thereby melt the powder and obtain 50 g of a molten salt bath. Thus, in the same glove box as above, a nickel plate (The Nilaco Corporation; 99.7%, 10 mm in length×5 mm in width×0.2 mm in thickness) as the working electrode (cathode), a coil-like zinc wire (The Nilaco Corporation; 99.99%, 1 mm in diameter×50 mm in length) as the counter electrode, and a zinc wire (The Nilaco Corporation; 99.99%, 1 mm in diameter×5 mm in length) as the reference electrode, were immersed in the molten salt bath. Then, while keeping the molten salt bath at a temperature of 150° C., the potential of the working electrode was maintained at 100 mV (vs. Zn²⁺/Zn) to effect potentiostatic electrolysis for 3 hours. On observing the deposition product on the surface of the nickel plate used as the working electrode with a scanning electron microscope (SEM), the deposition product was confirmed to be a film-like product having excellent adhesion with the nickel plate. Furthermore, the deposition product was confirmed to be metallic tungsten on analyzing the deposition product using XPS (the details of the experimental conditions and the experimental results are given in Table 3). As is clearly from Table 3, it has been found that metallic tungsten of high purity can be deposited in a film-like form by using the molten salt bath.

### Experiment B:

Potentiostatic electrolysis was conducted in the same manner as in Experiment A, except for using 0.5 mol of tungsten trioxide (WO₃) in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic tungsten on analyzing the deposition product using XPS (the details of the experimental conditions and the experimental results are given in Table 3). As is clearly from Table 3, it has been found that metallic tungsten of high purity can be deposited in a film-like form by using the molten salt bath.

### Experiment C:

Potentiostatic electrolysis was conducted in the same manner as in Experiment A, except for using TriMePeAmCl and ZnCl₂ at a molar ratio of 40:60, and for using 1 mol of tantalum pentachloride (TaCl₅) in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic tantalum on analyzing the deposition product using XPS (the details of the experimental conditions and the experimental results are given in Table 3). As is clearly from Table 3, it has been found that metallic tantalum of high purity can be deposited in a film-like form by using the molten salt bath.

### Experiment D:

Potentiostatic electrolysis was conducted in the same manner as in Experiment A, except for using TriMePeAmCl and ZnCl₂ at a molar ratio of 60:40, and for using 1 mol of potassium tantalum fluoride (K₂TaF₇) in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic tantalum on analyzing the deposition product using XPS (the details of the experimental conditions and the experimental results are given in Table 3). As is clearly from Table 3, it has been found that metallic tantalum of high purity can be deposited in a film-like form by using the molten salt bath.

### Experiment E:

EtMePyrCl was dried in vacuum at 120° C. for 24 hours. Further, ZnCl₂ and KF were dried in vacuum at 200° C. for 24 hours. EtMePyrCl and ZnCl₂ were weighed at a molar ratio of 50:50 in a glove box under argon atmosphere, and were placed inside an alumina crucible. Then, 2 mol of KF (which approximately corresponds to the saturation amount of the dissolution of the compound in the molten salt) and 0.5 mol of WCl₄ were weighed with respect to 100 mol of a mixture of EtMePyrCl and ZnCl₂, and were placed inside the alumina crucible into which EtMePyrCl and ZnCl₂ were placed as above. Subsequently, the above alumina crucible into which the raw material powder were placed was heated to 150° C. inside the same glove box as above to thereby melt the powder and obtain 50 g of a molten salt bath. Thus, in the same glove box as above, a nickel plate (The Nilaco Corporation; 99.7%, 10 mm in length×5 mm in width×0.2 mm in thickness) as the working electrode (cathode), a coil-like zinc wire (The Nilaco Corporation; 99.99%, 1 mm in diameter×50 mm in length) as the counter electrode, and a zinc wire (The Nilaco Corporation; 99.99%, 1 mm in diameter×5 mm in length) as the reference electrode, were immersed in the molten salt bath. Then, while keeping the molten salt bath at a temperature of 150° C., the potential of the working electrode was maintained at 100 mV (vs. Zn²⁺/Zn) to effect potentiostatic electrolysis for 3 hours. On observing the deposition product on the surface of the nickel plate used as the working electrode with an SEM, the deposition product was confirmed to be a film-like product having excellent adhesion with the nickel plate. Furthermore, the deposition product was confirmed to be metallic tungsten on analyzing the deposition product using XPS (the details of the experimental conditions and the experimental results are given in Table 3). As is clearly from Table 3, it has been found that metallic tungsten of high purity can be deposited in a film-like form by using the molten salt bath.

### Experiment F:

Potentiostatic electrolysis was conducted in the same manner as in Experiment E, except for using 0.5 mol of WO₃ in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic tungsten on analyzing the deposition product using XPS (the details of the experimental conditions and the experimental results are given in Table 3). As is clearly from Table 3, it has been found that metallic tungsten of high purity can be deposited in a film-like form by using the molten salt bath.

### Experiment G:

Potentiostatic electrolysis was conducted in the same manner as in Experiment E, except for using 0.5 mol of molybdenum trichloride (MoCl₃) in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic molybdenum on analyzing the deposition product using XPS (the details of the experimental conditions and the experimental results are given in Table 3). As is clearly from Table 3, it has been found that metallic molybdenum of high purity can be deposited in a film-like form by using the molten salt bath.

### Experiment H:

Potentiostatic electrolysis was conducted in the same manner as in Experiment E, except for using 0.5 mol of molybdenum pentachloride (MoCl₅) in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic molybdenum on analyzing the deposition product using XPS (the details of the experimental conditions and the experimental results are given in Table 3). As is clearly from Table 3, it has been found that metallic molybdenum of high purity can be deposited in a film-like form by using the molten salt bath.

### Experiment I:

Potentiostatic electrolysis was conducted in the same manner as in Experiment E, except for using EtMePyrCl and ZnCl₂ at a molar ratio of 40:60, and for using 0.5 mol of MoCl₃ in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic molybdenum on analyzing the deposition product using XPS (the details of the experimental conditions and the experimental results are given in Table 3). As is clearly from Table 3, it has been found that metallic molybdenum of high purity can be deposited in a film-like form by using the molten salt bath.

### Experiment J:

Potentiostatic electrolysis was conducted in the same manner as in Experiment E, except for using EtMePyrCl and ZnCl₂ at a molar ratio of 40:60, and for using 0.5 mol of MoCl₅ in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic molybdenum on analyzing the deposition product using XPS (the details of the experimental conditions and the experimental results are given in Table 3). As is clearly from Table 3, it has been found that metallic molybdenum of high purity can be deposited in a film-like-form by using the molten salt bath.

### Experiment K:

Potentiostatic electrolysis was conducted in the same manner as in Experiment E, except for using 1 mol of titanium tetrachloride (TiCl₄) in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic titanium on analyzing the deposition product using XPS (the details of the experimental conditions and the experimental results are given in Table 3). As is clearly from Table 3, it has been found that metallic titanium of high purity can be deposited in a film-like form by using the molten salt bath.

### Experiment L:

Potentiostatic electrolysis was conducted in the same manner as in Experiment E, except for using 0.5 mol of niobium pentachloride (NbCl₅) in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic niobium on analyzing the deposition product using XPS (the details of the experimental conditions and the experimental results are given in Table 3). As is clearly from Table 3, it has been found that metallic niobium of high purity can be deposited in a film-like form by using the molten salt bath.

### Experiment M:

Potentiostatic electrolysis was conducted in the same manner as in Experiment E, except for using 0.5 mol of vanadium dichloride (VCl₂) in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic vanadium on analyzing the deposition product using XPS (the details of the experimental conditions and the experimental results are given in Table 3). As is clearly from Table 3, it has been found that metallic vanadium of high purity can be deposited in a film-like form by using the molten salt bath.

### Experiment N:

Potentiostatic electrolysis was conducted in the same manner as in Experiment E, except for using 0.5 mol of zirconium dichloride (ZrCl₂) in the place of 0.5 mol of WCl₄. On observing the deposition product on the surface of the nickel plate used as the working electrode with an SEM, the deposition product was confirmed to be a film-like product having excellent adhesion with the nickel plate. Furthermore, the deposition product was confirmed to be metallic zirconium on analyzing the deposition product using XPS (the details of the experimental conditions and the experimental results are given in Table 3). As is clearly from Table 3, it has been found that metallic zirconium of high purity can be deposited in a film-like form by using the molten salt bath.

TABLE 3

| | Composition of molten salt and raw material | | | | | | Condition of electrolysis | | | | | | | |
|---|---|---|---|---|---|---|---|---|---|---|---|---|---|---|
| | for electrodepositon of metal (molar ratio) | | | | | | (Potentiostatic electrolysis) | | | Deposition product *others | | | | |
| Ex. | TriMe | EtMe | | | Raw | | | | Time | | | | | |
| No. | PeAmCl | PyrCl | ZnCl₂ | KF | material | | Temperature | Potential | (h) | Form | Composition (atomic %) | | | |
| A | 50 | 0 | 50 | 2 | 0.5 | (WCl₄) | 150° C. | 100 mV | 3 | Film | 98.1 | (W) | 1.2 (O) | 0.7* |
| B | 50 | 0 | 50 | 2 | 0.5 | (WO₃) | ″ | ″ | ″ | ″ | 99.2 | (W) | 0.3 (O) | 0.5* |
| C | 40 | 0 | 60 | 2 | 1 | (TaCl₅) | ″ | ″ | ″ | ″ | 98.2 | (Ta) | 1.1 (O) | 0.7* |
| D | 60 | 0 | 40 | 2 | 1 | (K₂TaF₇) | ″ | ″ | ″ | ″ | 97.5 | (Ta) | 1.5 (O) | 1.0* |
| E | 0 | 50 | 50 | 2 | 0.5 | (WCl₄) | ″ | ″ | ″ | ″ | 98.3 | (W) | 0.9 (O) | 0.8* |
| F | 0 | 50 | 50 | 2 | 0.5 | (WO₃) | ″ | ″ | ″ | ″ | 99.3 | (W) | 0.4 (O) | 0.3* |
| G | 0 | 50 | 50 | 2 | 0.5 | (MoCl₃) | ″ | ″ | ″ | ″ | 98.8 | (Mo) | 0.6 (O) | 0.6* |
| H | 0 | 50 | 50 | 2 | 0.5 | (MoCl₅) | ″ | ″ | ″ | ″ | 99.1 | (Mo) | 0.4 (O) | 0.5* |
| I | 0 | 40 | 60 | 2 | 0.5 | (MoCl₃) | ″ | ″ | ″ | ″ | 98.2 | (Mo) | 0.9 (O) | 0.9* |
| J | 0 | 40 | 60 | 2 | 0.5 | (MoCl₅) | ″ | ″ | ″ | ″ | 99.2 | (Mo) | 0.4 (O) | 0.4* |
| K | 0 | 50 | 50 | 2 | 1 | (TiCl₄) | ″ | ″ | ″ | ″ | 97.2 | (Ti) | 2.1 (O) | 0.7* |
| L | 0 | 50 | 50 | 2 | 0.5 | (NbCl₅) | ″ | ″ | ″ | ″ | 98.3 | (Nb) | 1.2 (O) | 0.5* |
| M | 0 | 50 | 50 | 2 | 0.5 | (VCl₂) | ″ | ″ | ″ | ″ | 97.7 | (V) | 1.5 (O) | 0.8* |
| N | 0 | 50 | 50 | 2 | 0.5 | (ZrCl₂) | ″ | ″ | ″ | ″ | 97.4 | (Zr) | 1.6 (O) | 1.0* |

### INDUSTRIAL APPLICABILITY

The present invention has industrial applicability in the point that it provides an electrodeposition method for metals using a molten salt, which easily enables the electrodeposition of various types of metals such as refractory metals and rare earth metals.

1. An electrodeposition method for metals using a molten salt, which is characterized in that it is effected at the electrodeposition temperature in a range of from 100° C. to 200° C. using a molten salt of quaternary ammonium halide represented by the general formula (I) below (wherein, in the formula, R¹, R², R³, and R⁴, which may be the same or different from each other and may have a substituent, each represents an alkyl group having 1 to 12 carbon atoms or a cycloalkyl group having 5 to 7 carbon atoms; and X⁻ represents a halide anion which is a counter-ion of quaternary ammonium cation) and/or a molten salt of pyrrolidinium halide represented by the general formula (II) below (wherein, in the formula, R⁵ and R⁶, which may be the same or different from each other and may have a substituent, each represents an alkyl group having 1 to 12 carbon atoms or a cycloalkyl group having 5 to 7 carbon atoms; and X⁻ represents a halide anion which is a counter-ion of pyrrolidinium cation).
[Chemical 1]
R¹R²R³R⁴N⁺X⁻ (I)

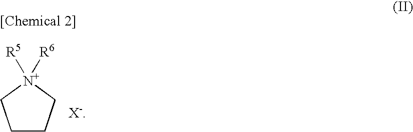

2. The method as claimed in claim 1, which is characterized in that the halide anion is a chloride ion.

3. The method as claimed in claim 1, which is characterized in that a metal halide compound is dissolved in the molten salt.

4. The method as claimed in claim 3, which is characterized in that the metal halide compound is at least one type selected from the group consisting of zinc chloride, tin chloride, and iron chloride.

5. The method as claimed in claim 3, which is characterized in that 0.5 mol to 2 mol of the metal halide compound is dissolved in 1 mol of the molten salt.

6. The method as claimed in claim 1, which is characterized in that an alkali metal chloride and/or an alkali metal fluoride is added in the molten salt.

7. The method as claimed in claim 1, which is characterized in that the electrodeposition temperature is in a range of from 130° C. to 180° C.

8. The method as claimed in claim 1, which is characterized in that the object metal intended for the electrodeposition is at least one type selected from a group consisting of refractory metals having a melting point of 1500° C. or higher, rare earth metals, and alloys containing at least one metal thereof.

9. A molten salt of pyrrolidinium halide characterized in that it is represented by the general formula (II) below (wherein, in the formula, R⁵ and R⁶, which may be the same or different from each other and may have a substituent, each represents an alkyl group having 1 to 12 carbon atoms or a cycloalkyl group having 5 to 7 carbon atoms; and X⁻ represents a halide anion which is a counter-ion of pyrrolidinium cation). [chemical structure]
